(19) United States
(12) Patent Application Publication
(10) Pub. No.: US 2006/0211691 A1
(43) Pub. Date: Sep. 21, 2006

(54) Use of 2h-[1,3]-oxazino[3,2-a] indole derivatives for the treatment of neuropathic pain

(75) Inventors: Angelo Guglielmotti, Roma (IT); Lorenzo Polenzani, Grottaferrata (IT); Alessandra Alisi, Roma (IT); Nicola Cazzolla, Albano Laziale (IT)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: AZIENDE CHIM. RIUN. ANG. FRANC. A.C.R.A.F. S.P.A., Roma (IT)

(21) Appl. No.: 10/560,836

(22) PCT Filed: Jul. 8, 2004
(86) PCT No.: PCT/EP04/07633

§ 371 Date: Mar. 30, 2006

(30) Foreign Application Priority Data
Jul. 18, 2005 (IT) MI2003A001467

(51) Int. Cl. A61K 31/538

(52) U.S. Cl. 514/230.2

(57) ABSTRACT

Use of a compound of formula (I), wherein R is H, a linear or branched alkyl chain having from 1 to 12 carbon atoms or an arylalkyl group, and of the acid addition salts thereof with pharmaceutically acceptable organic or inorganic acids, to prepare a pharmaceutical composition active in the treatment of neuropathic pain.

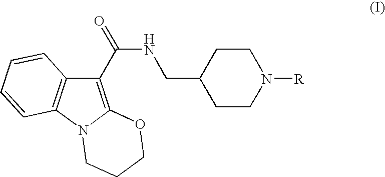

The present invention relates to the use of an indole compound for the preparation of a pharmaceutical composition active in the treatment of neuropathic pain.

European patent application EP-A-0 630 376 relates to a large number of compounds of formula I: 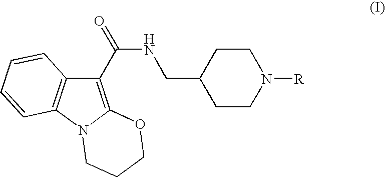
including those wherein
R is H, a linear or branched alkyl chain having from 1 to 12 carbon atoms or an arylalkyl radical.

According to the aforesaid document the compound of formula (I) is active in the treatment or the prophylaxis of gastrointestinal, cardiac and central nervous system disorders.

Hereinafter, the compounds of formula (I) wherein R has the aforesaid meanings will for brevity be referred to as “Compound (I)”.

It has now surprisingly been found that Compound (I) is particularly active in neuropathic pain.

It is known that on average about 10-20% of the adult population suffer from chronic pain. The chronic pain is generally associated with clinical conditions characterised by chronic and/or degenerative lesions.

Typical examples of pathological conditions characterised by chronic pain are rheumatoid arthritis, osteoarthritis, fibromyalgia, neuropathy, and the like [Ashburn M A, Staats P S, Management of chronic pain. Lancet 1999; 353: 1865-69].

Chronic pain, in particular neuropathic pain, is often debilitating and is a cause of loss of working capacity and poor quality of life. Consequently, it also results in economic and social losses.

The analgesic drugs currently used in the treatment of neuropathic pain include non-steroidal anti-inflammatories (NSAIDs), antidepressants, opioid analgesics, and anticonvulsants [Woolf C J, Mannion R J. Neuropathic pain: aetiology, symptoms, mechanism, and management. Lancet 1999; 353: 1959-1964].

However, chronic pain and, in particular, neuropathic pain is notoriously difficult to treat with the drugs currently available. Consequently, the development of novel analgesics has always been one of the major targets of the pharmaceutical industry. Moreover, in spite of the many research efforts intended to identify a suitable analgesic compound, there are a significant number of patients for whose pain condition there is still no satisfactory treatment [Scholz J, Woolf C J. Can we conquer pain? Nat Neusci. 2002; 5: 1062-76].

It is an object of the present invention to use a compound of formula (I), wherein R is H, a linear or branched alkyl chain having from 1 to 12 carbon atoms or an arylalkyl group and of the acid addition salts thereof with pharmaceutically acceptable organic or inorganic acids, to prepare a pharmaceutical composition active in the treatment of neuropathic pain.

Preferably, in the arylalkyl group the alkyl moiety has from 1 to 4 carbon atoms and the aryl moiety is a phenyl or naphthyl ring.

Typical examples of pharmaceutically acceptable organic or inorganic acids are: oxalic, maleic, methanesulphonic, paratoluenesulphonic, succinic, citric, tartaric, lactic, hydrochloric, phosphoric and sulphuric.

Typical examples of pathological conditions characterised by neuropathic pain are diabetes, cancer, immunodeficiency, traumas, ischaemia, multiple sclerosis, sciatic neuralgia, trigeminal neuralgia and post-herpetic syndromes.

Preferably, the pharmaceutical compositions of the present invention are prepared in the form of suitable dosage forms containing an effective dose of at least one Compound (I) or of an acid addition salt thereof with a pharmaceutically acceptable organic or inorganic acid and at least one pharmaceutically acceptable inert ingredient.

Examples of suitable dosage forms are tablets, capsules, coated tablets, granules, solutions and syrups for oral administration; medicated plasters, solutions, pastes, creams and ointments for transdermal administration; suppositories for rectal administration and sterile solutions for administration by the injection or aerosol routes.

Other suitable dosage forms are the sustained release dosage forms and the dosage forms based on liposomes for oral or injection administration.

The dosage forms may also comprise other conventional ingredients such as: preservatives, stabilisers, surfactants, buffers, salts to regulate the osmotic pressure, emulsifiers, sweeteners, colorants, flavourings and the like.

If required by particular therapies, the pharmaceutical composition of the present invention may comprise other pharmacologically active ingredients whose concomitant administration is useful.

The amount of Compound (I) or of an acid addition salt thereof with a pharmaceutically acceptable organic or inorganic acid in the pharmaceutical composition of the present invention can vary over a wide range depending on known factors such as, for example, the type of pathology with which the neuropathic pain to be treated is associated, the severity of the disease, the patient's body weight, the dosage form, the chosen administration route, the number of administrations per day and the efficacy of the chosen compound of formula (I). However, the optimal amount can be determined in a simple and routine manner by the person skilled in the art.

Typically, the amount of Compound (I) or of an acid addition salt thereof with a pharmaceutically acceptable organic or inorganic acid in the pharmaceutical composition of the present invention will be such as to ensure an administration level of from 0.001 to 100 mg/kg/day of Compound (I), as a base. Preferably, the administration level will be of from 0.05 to 50 mg/kg/day, and still more preferably of from 0.1 to 10 mg/kg/day.

The dosage forms of the pharmaceutical composition of the present invention can be prepared by techniques well known to the pharmaceutical chemist which include mixing, granulating, compressing, dissolving, sterilizing and the like.

The analgesic activity of Compound (I) has been proved by means of two experimental models in the rat: allodynia induced by ligature of the sciatic nerve and mechanical hyperalgesia in diabetic neuropathy induced by streptozotocin.

As is known to the person skilled in the art, the aforesaid experimental models can be considered to be predictive of activity in man.

The experimental model of ligature of the sciatic nerve in the rat is a neuropathy which reproduces a series of responses similar to those observed in man in many traumatic and pathological conditions associated with neuropathic pain. Ligature of the sciatic nerve is in fact capable of inducing a syndrome associated with the activation of specific circuits responsible for the control of the perception of pain and characterised by the appearance of allodynia, hyperalgesia and spontaneous pain. As is well known, this model is an effective instrument for the study of drugs for use in the treatment of neuropathic pain in man and, in particular, in the control of conditions such as allodynia and hyperalgesia.

In its turn, the diabetic neuropathy induced by streptozotocin in the rat is an insulin-dependent syndrome characterised by a concomitant decrease in the conduction speed of the motor and sensory nerves and the appearance of a series of anomalies in the perception of pain. As is well known, this model is a useful instrument for the study of drugs for use in the treatment of neuropathic pain in man. In particular, the model is a valid example of a large group of neuropathic pain types characterised by phenomena such as hyperalgesia and allodynia due to primary lesions or dysfunctions of the nervous system.

Typical examples of human pathologies characterised by the dysfunctions described in the two experimental models cited above and characterised by the presence of neuropathic pain are diabetes, cancer, immunodeficiency, trauma, ischaemia, multiple sclerosis, sciatic neuralgia, trigeminal neuralgia and post-herpetic syndromes.

### Tests

1. Allodynia Induced by Ligature of the Sciatic Nerve in the Rat

Male CD rates of weight 200-250 g on arrival were used.

The allodynia was induced by ligature under anaesthesia of the sciatic nerve of the left hind paw [Seltzer Z, Dubner R, Shir Y. A novel behavioral model of neuropathic pain disorders produced in rats by partial sciatic nerve injury. Pain 1990; 43: 205-218; Bennett G J, Xie Y K. A peripheral mononeuropathy in rat that produces disorders of pain sensation like those seen in man. Pain 1998; 33: 87-107]. After at least two weeks following the ligature of the sciatic nerve, rats which showed a reduction of a least 50% in the response threshold recorded before the operation were selected. The pain threshold was measured by means of a von Frey instrument which, by applying a gradual increase in pressure on the plantar zone of the left hind paw of the rat, makes it possible to record the nocifensive response, expressed in grams, corresponding to the moment at which the animal withdraws its paw.

At 30 minutes, 1, 2 and 4 hrs after the treatment, the pain threshold measured in control animals was compared with that measured in animals treated with the compound under test.

The control animals were treated with same vehicle (water) as was used for administration of the compound under test (hydrochloride salt of the compound of formula (I) wherein R is n-butyl, prepared as disclosed in Example 3, Method 2, of EP-A-0 630 376).

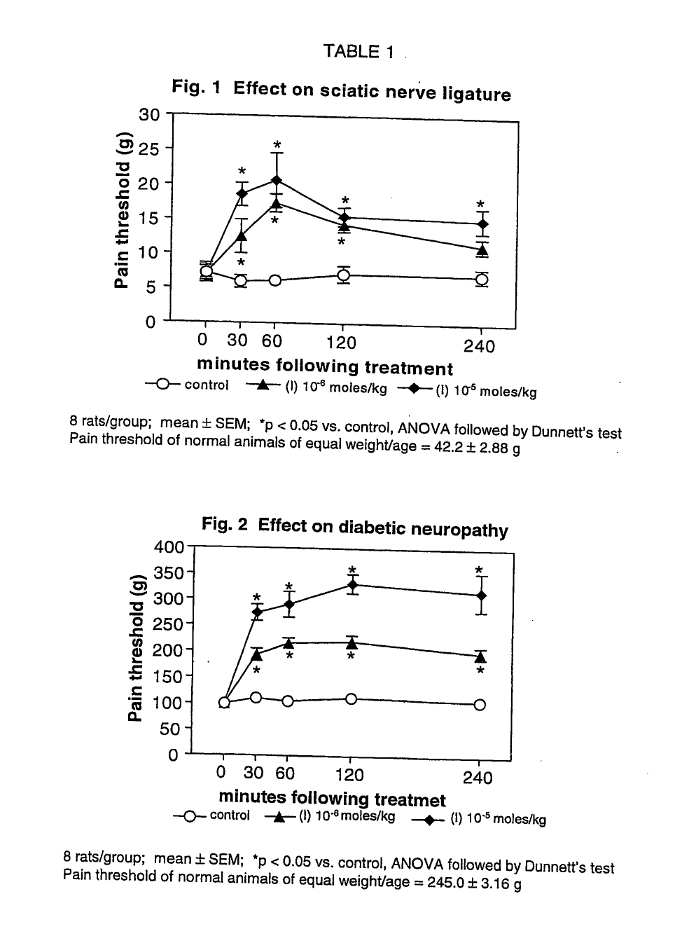

The results are shown in FIG. 1.

Similar results were obtained with the hydrochloride salt of the compound of formula (I), wherein R is cyclohexyl, prepared according to Examples 23 of EP-A-0 630 376.

2. Mechanical Hyperalgesia in Rats with Diabetes Induced by Streptozotocin

Male CD rates of weight 240-300 g on arrival were used.

The diabetic syndrome was induced by a single intraperitoneal (i.p.) injection of 80 mg/kg of streptozotocin dissolved in sterile physiological solution [Courteix C, Eschalier A, Lavarenne J. Streptozotocin-induced diabetic rats: behavioural evidence for a model of chronic pain. Pain, 1993; 53: 81-88; Bannon A W, Decker M W, Kim Dj, Campbell J E, Arneric S P. ABT-594, a novel cholinergic channel modulator, is efficacious in nerve ligation and diabetic neuropathy models of neuropathic pain. Brain Res. 1998; 801: 158-63].

After at least three weeks following the injection of streptozotocin, rats with a glycaemia level ≧300 mg/dl and a response threshold ≦120 g to a mechanical nociceptive stimulus were selected. The glycaemia levels were measured using a reflectometer utilising reactive strips impregnated with glucose oxidase. The pain threshold was measured using an analgesimeter. The instrument, by applying a gradual increase in pressure on the dorsal zone of the left hind paw of the rat, makes it possible to record the nocifensive response, expressed in grams, corresponding to the moment at which the animal withdraws its paw.

At 30 minutes, 1, 2 and 4 hrs after the treatment, the pain threshold measured in control animals was compared with that measured in animals treated with the compound under test.

The control animals were treated with same vehicle (water) as was used for administration of the compound under test (hydrochloride salt of the compound of formula (I) wherein R is n-butyl, prepared as disclosed in Example 3, Method 2, of EP-A-0 630 376).

The results are shown in FIG. 2.

Similar results were obtained with the hydrochloride salt of the compound of formula (I), wherein R is cyclohexyl, prepared according to Examples 23 of EP-A-0 630 376.

### EXAMPLES

### Example 1

A tablet comprising, as the active principle, the Compound (I) of the present invention, has the following composition:

| | |
|---|---|
| Active principle | 50 mg |
| Lactose monohydrate | 161 mg |
| Dibasic calcium phosphate dihydrate | 161 mg |
| Microcrystalline cellulose | 95 mg |
| Maize starch | 30 mg |
| Sodium carboxymethyl starch | 24 mg |
| Povidone | 11 mg |
| Magnesium stearate | 3 mg |

### Example 2

An ampoule comprising, as the active principle, the Compound (I) of the present invention, has the following composition:

Active principle 25 mg

Sorbitol q.s. for isosmotic solution

Water q.s to 100 ml

### Example 3

A pharmaceutical composition in granules comprising, as the active principle, a Compound (I) of the present invention, has the following composition:

| | |
|---|---|
| Active principle | 50 mg |
| Maltitol | 1300 mg |
| Mannitol | 2700 mg |
| Saccharose | 1000 mg |
| Citric acid | 20 mg |
| Aspartame | 20 mg |
| Flavourings | 200 mg |

1. A method for the treatment of neuropathic pain in a subject in need thereof comprising administering to the subject a pharmaceutical composition in an amount sufficient to treat neuropathic pain wherein the pharmaceutical composition comprises a compound of formula I: 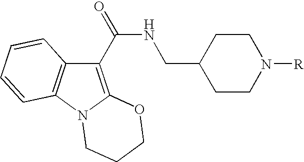
wherein
R is H, a linear or branched alkyl chain having from 1 to 12 carbon atoms or an arylalkyl group,
and of the acid addition salts thereof with pharmaceutically acceptable organic or inorganic acids.

2. The method according to claim 1, wherein R is an arylalkyl group where the alkyl moiety has from 1 to 4 carbon atoms.

3. The method according to claim 1, wherein R is an arylalkyl group where the aryl moiety is a phenyl or naphthyl ring.

4. The method according to claim 1, wherein R is an n-butyl group.

5. The method according to claim 1, wherein R is a cyclohexyl group.
